(19) United States
(12) Patent Application Publication
(10) Pub. No.: US 2019/0019142 A1
(43) Pub. Date: Jan. 17, 2019

(54) SYSTEM AND METHOD OF REVERSE MODELING OF PRODUCT TEMPERATURES

(71) Applicant: Carrier Corporation, Palm Beach Gardens, FL (US)

(72) Inventor: Jonathan Cherneff, Roslindale, MA (US)

(21) Appl. No.: 16/080,228

(22) PCT Filed: Feb. 28, 2017
(86) PCT No.: PCT/US2017/020005

§ 371 Date: Aug. 27, 2018

Related U.S. Application Data
(60) Provisional application No. 62/301,964, filed on Mar. 1, 2016.

(51) Int. Cl. G06Q 10/08; G01K 3/10; G01N 33/02; G01K 7/42; G01K 3/14

(52) U.S. Cl. CPC G06Q 10/0832; G01K 3/10; G01K 1/024; G01K 7/427; G01K 3/14; G01N 33/02

(57) ABSTRACT

A method of estimating a temperature history of a product transported in a shipping enclosure, the method including measuring an actual ending temperature of the product, acquiring ambient temperature data of the shipping enclosure, creating a plurality of simulated trips based in part on the ambient air temperature data of the shipping enclosure, wherein each of the plurality of simulated trips includes an estimated ending product temperature value, and determining whether the difference between the estimated ending product temperature value and the actual temperature of the product is within a temperature threshold.

### CROSS REFERENCE TO RELATED APPLICATION

The present application is an international patent application, which claims priority to U.S. Patent Application Ser. No. 62/301,964, filed Mar. 1, 2016, which is herein incorporated in its entirety.

### TECHNICAL FIELD OF THE DISCLOSED EMBODIMENTS

The presently disclosed embodiments generally relate to modeling temperatures, and more particularly, to a system and method reverse modeling of product temperatures

### BACKGROUND OF THE DISCLOSED EMBODIMENTS

Many products, such as refrigerated products, have temperature requirements. It can be prohibitively expensive in terms of both time and money to place thermometers within, or in contact with, the products themselves. Therefore, temperature measurements are typically taken of the surrounding ambient air. However, measuring the temperature of the ambient air can be a poor substitute for the information that is actually desired, i.e., the temperature of the product.

Moreover, the process of accepting or rejecting food shipments typically involves taking a temperature of the product via a temperature probe; however, that alone does not indicate the probable temperature history of the product. There is therefore a need for an improved method of modeling product temperature to determine whether shipments should be accepted or rejected.

### SUMMARY OF THE DISCLOSED EMBODIMENTS

In one aspect, a method of estimating a temperature history of a product transported in a shipping enclosure is provided. The method includes measuring an actual ending temperature of the product, acquiring ambient temperature data of the shipping enclosure, creating a plurality of simulated trips based in part on the ambient air temperature data of the shipping enclosure, wherein each of the plurality of simulated trips includes an estimated ending product temperature value, and determining whether the difference between the estimated ending product temperature value and the actual ending temperature of the product is within a temperature threshold. In one embodiment, the temperature threshold is adjustable.

In one embodiment, measuring an actual temperature of the product includes measuring the actual temperature of the product upon arrival at a destination other than the origin. In one embodiment, acquiring ambient temperature data of the shipping enclosure includes measuring ambient air temperature data within the shipping enclosure for a trip duration time. In one embodiment, creating a plurality of simulated trips based in part on the ambient air temperature data of the shipping enclosure includes determining at least one of a thermal mass and a thermal conductivity of the product.

In one aspect, a system for estimating a temperature history of a product transported in a shipping container is provided. The system includes a network, a first data recordation device in communication with the network, wherein the first data recordation device is configured to acquire ambient temperature data of the shipping enclosure, and a remote computing device in communication with the network, wherein the remote computing device is configured to create a plurality of simulated trips based in part on the ambient air temperature data of the shipping enclosure, wherein each of the plurality of simulated trips includes an estimated ending product temperature value.

In an embodiment, the system further includes a second data recordation device in communication with the network, wherein the second data recordation device is configured to measure an actual temperature of the product. In an embodiment of the system, the remote computing device is further configured to determine whether the difference between the estimated ending product temperature value and the actual temperature of the product is within a temperature threshold. In an embodiment, the temperature threshold is adjustable.

### BRIEF DESCRIPTION OF DRAWINGS

FIG. 1 illustrates a schematic diagram of a modeling system according to an embodiment of the present disclosure;

FIG. 2 illustrates a schematic flow diagram of a method to reverse model product temperatures according to an embodiment of the present disclosure;

FIG. 3 illustrates a graph depicting a reverse model of product temperatures according to an embodiment of the present disclosure; and

FIG. 4 illustrates a graph depicting a reverse model of product temperatures according to an embodiment of the present disclosure.

### DETAILED DESCRIPTION OF THE DISCLOSED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

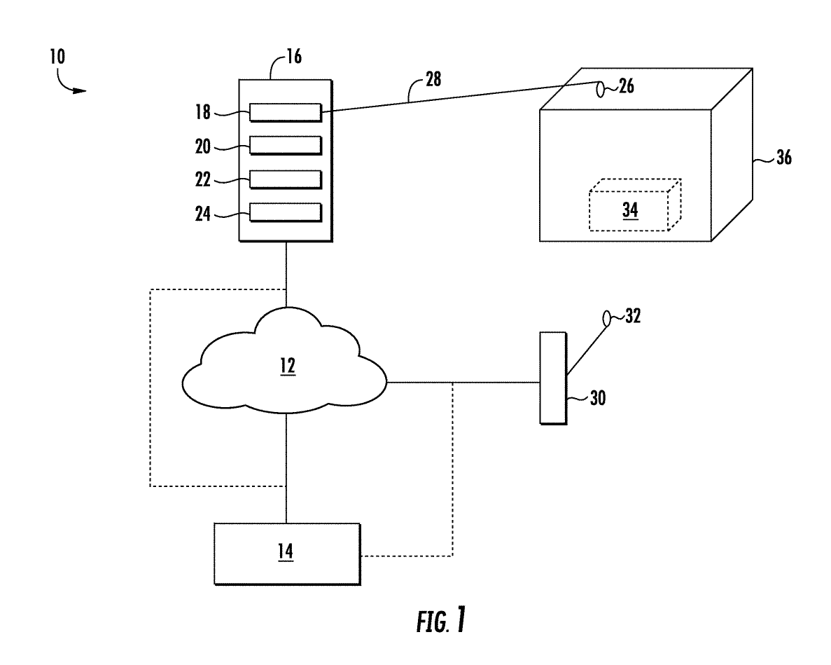

FIG. 1 schematically illustrates an embodiment of a product temperature measuring system, generally indicated at 10. In the embodiment shown, the system 10 includes a network 12 in communication with a remote computing device 14 and a first data recordation device 16. It will be appreciated that the remote computing device 14 may be in direct communication with the first data recordation device 16 via a wired or wireless connection.

The data recordation device 16 includes an intake engine 18, a calculation engine 20, an interface engine 22, a database 24, and a first temperature measuring device 26 communicatively connected with intake engine 18 via a wire 28. It will be appreciated that the first temperature measuring device 26 may be communicatively connected to the data recordation device 16 via any means known in the art, such as wirelessly. The data recordation device 16 is configured to measure an ambient air temperature within a shipping enclosure (not shown), for example, a refrigerated truck to name one non-limiting example. In the embodiment shown, the first temperature measuring device 26 is placed on a product container 36 within the shipping enclosure. It will be appreciated that the first temperature measuring device 26 may be placed anywhere within the shipping enclosure suitable to measure the ambient air temperature therein.

The system 10 further includes a second data recordation device 30 in communication with the network 12. The second data recordation device 30 includes a second temperature measuring device 32, and is configured to measure an actual temperature of a product 34 stored and transported in a product container 36. The second data recordation device 30 is typically located at one or more of the locations along the route of the shipping enclosure. It will be appreciated that the remote computing device 14 may be in direct communication with the second data recordation device 30 via a wired or wireless connection. The system 10 is configured to estimate a temperature history of the product 34 stored and transported in the product container 36 based in part on the method described herein.

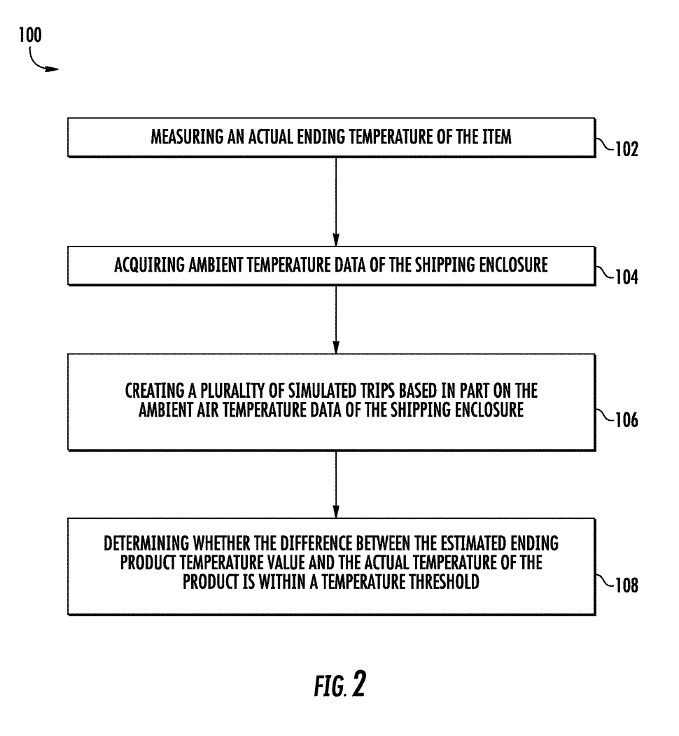

FIG. 2 illustrates a method of estimating a temperature history of the product 34 stored and transported in the shipping enclosure, the method generally indicated at 100. The method 100 includes step 102 of measuring an actual temperature of the product 34. In an embodiment, measuring an actual temperature of the product 34 includes measuring the actual temperature of the product 34 upon arrival at a destination other than the origin. With reference to FIG. 1, upon arrival at a destination, for example a final destination, of the shipment of product 34, a user may measure the temperature of the product 34 utilizing the second temperature measuring device 32 of the second data recordation device 30.

The method 100 further includes step 104 of acquiring ambient temperature data of the shipping enclosure. In an embodiment, step 104 includes operating the first data recordation device 16 to measure ambient air temperature data within the shipping enclosure for a trip duration time.

The method 100 further includes step 106 of creating a plurality of simulated trips based in part on the ambient air temperature data of the shipping enclosure, wherein each of the plurality of simulated trips includes an estimated ending product temperature value. In an embodiment, step 106 further includes determining at least one of a thermal mass, a thermal conductivity, and thermal characteristics of the product 34. For example, a user may operate the remote computing device 14 to select the type of product 34 being transported, for example, a specific type of meat, produce, ice cream, etc. to name a few non-limiting examples. The remote computing device 14 may either store in memory or download from the network 12 the thermal mass and/or thermal conductivity and/or other thermal characteristics of the product 34.

The method 100 further includes step 108 of determining whether the difference between the estimated ending product temperature value and the actual temperature of the product 34 is within a temperature threshold. In one embodiment, the temperature threshold is adjustable.

Working through an example, for illustrative purposes only, before the product 34 leaves its origin, a user may operate the first data recordation device 16 to begin collecting ambient air temperature data within the shipping enclosure. During the duration of the trip, the first data recordation device 16 measures and records the ambient air temperature within the shipping enclosure. Once the product 34 arrives at the desired destination, another user may operate the first data recordation device 16 to transmit the ambient air temperature data for the duration of the trip to the remote computing device 14 over the network 12.

In an embodiment, once the remote computing device 14 receives the ambient air temperature data from the first data recordation device 16 and the actual temperature of the product 34 upon arrival, the remote computing device 14 selects an estimated starting product temperature value to begin a simulated trip. Based on the estimated starting product temperature value, the remote computing device 14 utilizes the following calculation to produce an estimated temperature difference between the product and the ambient air.

ΔTₙ₊₁=k(Aₙ−Tₙ)

Where Aₙ is the ambient air temperature value at a given time, Tₙ, is the estimated temperature of the product at the given time, k is the thermal conductivity and/or the thermal mass of the product 34.

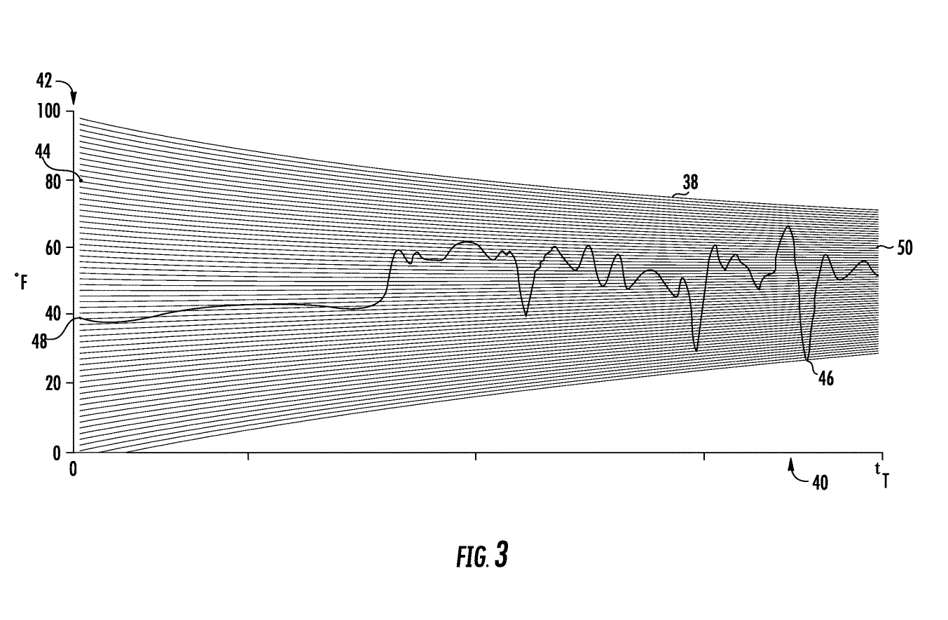

An example of a plurality of simulated trips 38 is shown in FIG. 3. The x-axis 40 of the graph is time, and the y-axis 42 is the estimated temperature of the product 34 in degrees Fahrenheit (° F.). To estimate the temperature history of the product 34, for illustrative purposes only, the remote computing device 14 first selects an estimated starting product temperature value, for example approximately 80° F. (designated at point 44). Assuming a thermal conductivity and thermal mass value k of approximately 0.0375, the remote computing device 14 takes the difference between the ambient air temperature value, as shown by the line 46, and the estimated starting product temperature value at the next time interval (i.e., t₁).

In the example shown, the ambient air temperature value at time t₁, designated at point 48 is approximately 39° F. As such, the remote computing device 14 then estimates the temperature difference of the product 34 at time t₂ to be approximately −1.5375° F., resulting in an estimated product temperature at time t₂ of approximately 78.4625° F. The remote computing device 14 will continue to estimate the temperature of the product 34 for the time period equivalent to the entire duration of the trip until an estimated ending product temperature is determined (e.g. approximately 63° F. for a starting temperature of approximately 80° F.).

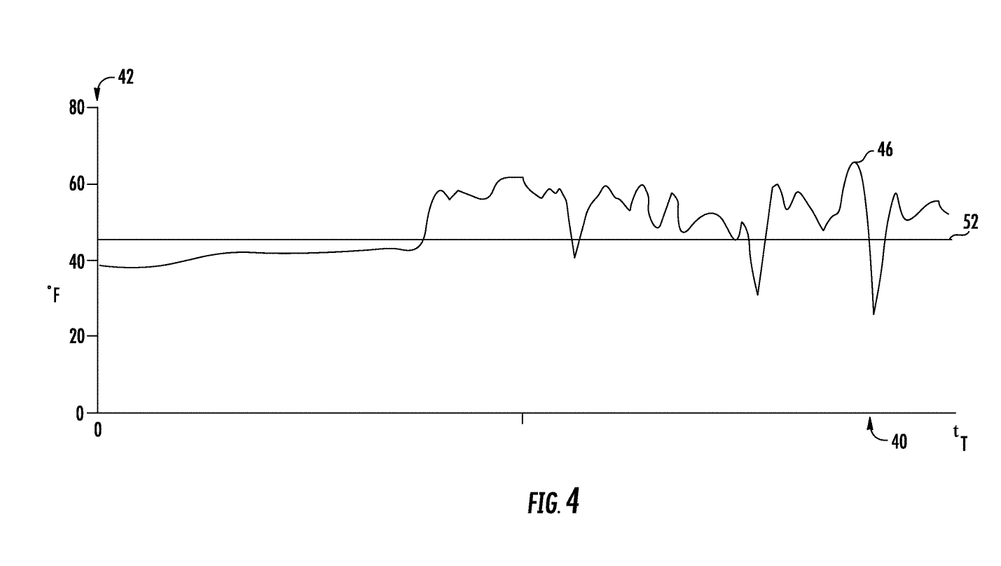

If the actual temperature of the product 34 is measured at approximately 44° F. at the end of the trip, and the temperature threshold is established as ±1° F., the remote computing device 14 determines which of the simulated trips produces an estimated ending product temperature of approximately 44° F.±1° F. As shown in FIG. 4, the remote computing device determines that the simulated trip 52 produces an estimated ending product temperature of approximately 44° F.±1° F. As such, a user may examine the simulated trip 38′ to estimate the temperature history of the product 34 for the duration of trip.

It will therefore be appreciated that the present system 10 and method 100 allows a user to estimate the temperature history of the product 34 by using the ambient air temperature data, the actual temperature of the product 34 upon arrival at the final destination; thus providing the user with valuable information and allowing the user to make better judgments on the acceptance or denial of the product based on the temperature history of the product 34.

While the disclosure has been illustrated and described in detail in the drawings and foregoing description, the same is to be considered as illustrative and not restrictive in character, it being understood that only certain embodiments have been shown and described and that all changes and modifications that come within the spirit of the disclosure are desired to be protected.

What is claimed is:

1. A method of estimating a temperature history of a product transported in a shipping enclosure, the method comprising:
(a) measuring an actual ending temperature of the product;
(b) acquiring ambient temperature data of the shipping enclosure;
(c) creating a plurality of simulated trips based in part on the ambient air temperature data of the shipping enclosure, wherein each of the plurality of simulated trips includes an estimated ending product temperature value; and
(d) determining whether the difference between the estimated ending product temperature value and the actual ending temperature of the product is within a temperature threshold.

2. The method of claim 1, wherein step (a) comprises measuring the actual temperature of the product upon arrival at a destination other than the origin.

3. The method of claim 1, wherein step (b) comprises measuring ambient air temperature data within the shipping enclosure for a trip duration time.

4. The method of claim 1, wherein step (c) further comprises determining at least one of a thermal mass and a thermal conductivity of the product.

5. The method of claim 1, wherein the temperature threshold is adjustable.

6. A system for estimating a temperature history of a product transported in a shipping container comprising:
a network;
a first data recordation device in communication with the network, wherein the first data recordation device is configured to acquire ambient temperature data of the shipping enclosure; and
a remote computing device in communication with the network, wherein the remote computing device is configured to create a plurality of simulated trips based in part on the ambient air temperature data of the shipping enclosure, wherein each of the plurality of simulated trips includes an estimated ending product temperature value.

7. The system of claim 6, further comprising a second data recordation device in communication with the network, wherein the second data recordation device is configured to measure an actual ending temperature of the product.

8. The system of claim 7, wherein the remote computing device is further configured to determine whether the difference between the estimated ending product temperature value and the actual ending temperature of the product is within a temperature threshold.

9. The system of claim 8, wherein the temperature threshold is adjustable.
